(19) United States
(12) Patent Application Publication
(10) Pub. No.: US 2021/0236850 A1
(43) Pub. Date: Aug. 5, 2021

(54) RADIOACTIVE SEED LOADING APPARATUS

(71) Applicant: GT Medical Technologies, Inc., Tempe, AZ (US)

(72) Inventors: John Baker, Gilbert, AZ (US); David Brachman, Phoenix, AZ (US)

(21) Appl. No.: 17/233,836

(22) Filed: Apr. 19, 2021

Related U.S. Application Data
(63) Continuation-in-part of application No. 16/275,941, filed on Feb. 14, 2019, now Pat. No. 10,981,018.

(51) Int. Cl. A61N 5/10

(52) U.S. Cl. CPC A61N 5/1007; A61N 2005/1009; A61N 2005/1012; A61N 2005/1023; A61N 2005/101

(57) ABSTRACT

A loading apparatus is usable to embed radioactive seeds into carriers, while limiting exposure of the user to radioactive energy from the radioactive seeds. The loading apparatus facilitates accurate positioning of radioactive seeds within a carrier. The illustrated loaders generally comprise two components, a base and a lid, although in other embodiments the loaders may be separated into additional components.

### FIELD

The invention generally relates to improvements to radioactive brachytherapy.

### BACKGROUND

Tumors in living organisms are highly variable in size, location and their amount of infiltration into normal tissues, and the variability of tumors in general make them very difficult to treat with a one-size fits all approach. Furthermore, the extent of tumors and/or void created upon debulking are typically not known until presented in the operating room. Thus, the options necessary to effectively treat a tumor or tumor bed need to be quite diverse.

Brachytherapy involves placing a radiation source either into or immediately adjacent to a tumor. It provides an effective treatment of cancers of many body sites. Brachytherapy, as a component of multimodality cancer care, provides cost-effective treatment. Brachytherapy may be intracavitary, such as when treating gynecologic malignancies; intraluminal, such as when treating esophageal or lung cancers; external surface, such as when treating cancers of the skin, or interstitial, such as when treating various central nervous system tumors as well as extracranial tumors of the head and neck, lung, soft tissue, gynecologic sites, liver, prostate, and skin.

### SUMMARY

The systems, methods, and devices described herein each have several aspects, no single one of which is solely responsible for its desirable attributes. Without limiting the scope of this disclosure, several non-limiting features will now be described briefly.

In one embodiment, a loading apparatus comprises a base having a loading bed configured to support a radioactive seed carrier configured to receive one or more radioactive seed, and a base loading channel configured to receive an injection device, the injection device configured to inject the radioactive seed into the radioactive seed carrier, The loading apparatus may comprise a lid configured to engage with the base to create a loading chamber with the loading bed as the lower surface, the lid having a spring-loaded push block configured to exert force towards the loading bed of the base, wherein when a radioactive seed carrier is positioned on the loading bed the spring-loaded push block secures the radioactive seed carrier.

In some embodiments, the loading apparatus further comprises a lid loading channel substantially co-axial with the base loading channel when the lid is engaged with the base.

In some embodiments, the loading apparatus further comprises one or more seed alignment windows positioned to provide visibility into the loading chamber where one or more of the radioactive seeds are positioned in the radioactive seed carrier.

In some embodiments, the loading apparatus further comprises one or more carrier alignment windows positioned to provide visibility into the loading chamber where the radioactive seed carrier is positioned.

### BRIEF DESCRIPTION OF THE DRAWINGS

The principles of the present invention will be apparent with reference to the following drawings, in which like reference numerals denote like components:

FIGS. 1A and 1B are perspective views of an example carrier in a “cold” carrier state and a “hot” carrier state, respectively.

FIG. 2 is a cross-sectional drawing illustrating a portion of patient tissue having a tumor bed therein.

FIG. 3 is a top view of a carrier sheet configured for implantation of multiple radioactive seeds.

FIGS. 4A-4C are perspective views of a loading apparatus that is usable to embed radioactive seeds into carriers, while limiting exposure of the user to radioactive energy from the radioactive seeds.

FIGS. 5A and 5B illustrates example strands that may be used, in conjunction with an insertion device, with the loaders discussed herein.

FIG. 6 is a perspective view of an insertion side of a loader, comprising a base engaged with a cover, and shown with a strand inserted through the guide channels.

FIGS. 7A and 7B illustrate exploded views of a loader, each including a cover, a base, and push block.

FIG. 8A illustrates engagement surfaces of a base.

FIG. 8B illustrates engagement surfaces of a cover.

FIG. 9A is a top view of a loader, with FIG. 9B illustrating a cross-section 9B of FIG. 9A and FIG. 9C illustrating cross section 9C of FIG. 9A.

FIGS. 10A-10B are distal end views of example loading devices, with varying quantities and positions of guide channels.

FIG. 10C is a proximal end view of an example loading device.

FIG. 10D is a perspective view of a proximal end of a loading device.

FIGS. 10E-10G are proximal end views of example loading devices that each include multiple rows of guide channels.

FIG. 11 is a perspective view of a loader base that may be used to retain spherical, or semi-spherical, carriers for insertion of radioactive seeds.

FIG. 12 is a proximal end view of a loading device configured for loading of radioactive seeds within a spherical, or hemispherical, carrier.

FIG. 13 is a perspective view of another example loading device base, which may be used in conjunction with a corresponding lid (not shown) to retain a carrier sheet (or other shape carrier) for insertion of one or more radioactive seeds.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

### Terms

To facilitate an understanding of the systems and methods discussed herein, several terms are described below. These terms, as well as other terms used herein, should be construed to include the provided descriptions, the ordinary and customary meanings of the terms, and/or any other implied meaning for the respective terms, wherein such construction is consistent with context of the term. Thus, the descriptions below do not limit the meaning of these terms, but only provide example descriptions.

Tumor: an abnormal growth of tissue resulting from uncontrolled, progressive multiplication of cells. Tumors can be benign or malignant.

Tumor bed: an anatomical area of a patient (e.g., a human or other mammal) where a tumor exists (pre-operative tumor bed) and/or an area surrounding a surgically removed tumor (post-operative tumor bed), such as a cranial cavity from which a tumor was surgically removed. Even after surgical removal of a tumor, the remaining tumor bed of the patient may include tumor cells.

Treatment area: an anatomical area that is targeted for delivery of radiation, such as from one or more radiation delivery devices (e.g., the carriers discussed below). A treatment area may include tissue below and/or around a location where the radiation deliver device is positioned, such as an anatomical area of a tumor or a tumor bed.

Treatment surface: an anatomical surface of a patient (e.g., a human or other mammal) where a radiation delivery device is to be placed to deliver radiation to a treatment area, such as the treatment surface itself and/or tissue below the treatment surface. A treatment surface may be a portion of a tumor bed or any other anatomical surface. For example, if a tumor bed is surgically created, the treatment surface may include an entire exposed surface of the tumor bed, a portion of such exposed surface, or the entire exposed surface of the tumor bed as well as a surrounding area of tissue.

Brachytherapy: radiation treatment in which the radiation delivery device is placed directly on and/or close to a treatment surface of the body, such as directly on the surface of the body, within the body, or in a tumor bed. For example, brachytherapy may be intracavitary, such as in cranial or gynecologic malignancies; intraluminal, such as in esophageal or lung cancers; external, such as in cancers of the skin; and/or interstitial, such as in treatment of various central nervous system tumors as well as extracranial tumors of the head, neck, lung, soft tissue, gynecologic sites, rectum, liver, prostate, and penis.

Seed: a radioactive material that is configured for delivery of radiation to a tumor and/or tumor bed. A seed may be in various shapes and sizes, such as cylinder, cone, sphere, pyramid, cube, prism, rectangular prism, triangular prism, and/or any combination of these or other shapes. While seeds are generally referred to herein as cylindrical, any other shape or size of seed may alternatively be used in the various systems and methods discussed herein. Seeds may comprise any combination of one or more of multiple radioactive components, such as Cs 131, Ir 192, I 125, Pd 103, for example. Seeds may include a protective outer shell that partially or fully encases the radioactive material. Seeds are one form of radiation source. The term “radiation source,” as used herein, generally refers to a radioactive seed (or other object that emits radiation), either alone (e.g., a seed) or embedded, or otherwise attached to, a carrier (e.g., a tile carrier with an embedded radioactive seed).

Carrier (also referred to as a “carrier sheet”): a substrate that holds or contains a radioactive seed. A carrier that contains one or more seeds is a radiation delivery device. Carriers may comprise various materials, such as one or more bioresorbable materials, such as collagen. Thus, these bioresorbable materials are biodegradable, or naturally absorbing into the mammalian tissue over time, such as over a period of weeks or months. Carriers may be configured for permanent implantation into a tumor bed, such as to provide radioactive energy to a treatment surface surrounding an area where a tumor has been removed in order to treat any remaining malignant tissue. Carriers can be composed of various materials and take on various shapes and sizes. Examples carriers, such as carriers having various sizes, shapes, configurations, etc., are included in the following patent and patent application, each of which is hereby incorporated by reference in its entirety and for all purposes:
U.S. patent application Ser. No. 14/322,785, filed Jul. 2, 2014, now U.S. Pat. No. 8,876,684, entitled “Dosimetrically Customizable Brachytherapy Carriers and Methods Thereof In The Treatment Of Tumors,” and
U.S. patent application Ser. No. 14/216,723, filed Mar. 17, 2014, now U.S. Pat. No. 9,492,683, entitled “Dosimetrically Customizable Brachytherapy Carriers and Methods Thereof In The Treatment Of Tumors.”
Co-pending U.S. Provisional Application No. 63/163,583, filed Apr. 16, 2021, entitled “Custom Brachytherapy Carriers.”
Co-pending U.S. Provisional Application No. 63/163,366, filed Mar. 19, 2021, entitled “Systems and Methods For Creating Custom Brachytherapy Carriers.”

Tile Carrier (also referred to as “Tile”): type of carrier that is substantially planar and generally maintains a two-dimensional planar geometry when placed in a tumor bed. Tiles are generally rectangular cuboids (or other parallelepipeds), e.g., wherein all 6 sides are rectangular and generally planar. Depending on the material of the tile, though, the tile may be malleable such that the tile can be deformed by bending in order to better conform to a tumor bed. For example, for tiles comprising essentially collagen (and/or other malleable materials), the tiles may be substantially bent as placed in or on a treatment surface (and/or when pressed against the treatment surface) to conform with the shape of the treatment surface, such as a post-operative tumor bed.

Custom (or “non-planar”) Carrier: a carrier having one or more non-planar surfaces, such as a spherical shape or having a spherical portion. Examples of custom carriers include Spherical Carriers, Gore Carriers, and Star Carriers, noted below, as well as other custom carriers discussed herein.

Spherical Carrier (or “GammaSphere”): a substantially radially symmetrical body around an axis. A spherical carrier may also include a non-spherical portion, such as a tapered portion that extends from a spherical portion. Examples of other variations of spherical carriers are discussed herein.

Gore Carrier (also referred to as “Gore”): type of carrier that is 3-dimensional and conforms to the tumor bed while maintaining the geometry necessary for an effective implant. In some embodiments, gores are initially planar and are reconfigured to take on a 3-dimensional shape, such as to form a hemispherical surface that may be placed into a similarly shaped tumor cavity. Gore Carriers are further discussed in U.S. Pat. No. 8,876,684, entitled “Dosimetrically customizable brachytherapy carriers and methods thereof in the treatment of tumors,” filed on Jul. 2, 2014 as application Ser. No. 14/322,785, which is hereby incorporated by reference in its entirety and for all purposes.

Star Carrier (also referred to as “Star” or “arm-based carrier”): type of carrier that assumes a conformable 3-dimensional shape when arranged and placed into an operative cavity or similar space and conforms to the treatment environment while maintaining the geometry necessary for an effective implant. However, in some embodiments, Star carriers may be used in their initial planar state to cover a relatively flat tumor or tumor bed area. Star carriers are further discussed in U.S. Pat. No. 9,492,683, entitled “Dosimetrically customizable brachytherapy carriers and methods thereof in the treatment of tumors,” filed on Mar. 17, 2014 as application Ser. No. 14/216,723, which is hereby incorporated by reference in its entirety and for all purposes.

Loader (also referred to as a “loading device” or “loading apparatus”): a device that aids in placement of radioactive seeds in carriers, such as via injection of seeds into carriers. A loader may include multiple components, such as to hold a carrier in place and guide a delivery device (e.g., a needle or injector) into the carrier in order to place a seed at a precise location in the carrier. The “Loader Patents” refers to U.S. patent application Ser. No. 13/460,809, filed Apr. 30, 2012, now U.S. Pat. No. 8,939,881, entitled “Apparatus For Loading Dosimetrically Customizable Brachytherapy Carriers,” and U.S. patent application Ser. No. 14/696,293, filed Apr. 24, 2015, entitled “Apparatus and Method for Loading Radioactive Seeds Into Carriers,” which are each hereby incorporated by reference in their entirety for all purposes, describe several embodiments of loaders. As discussed further herein, loaders may be operated manually, such as by human operators, or may be fully automated, such that carriers can be loaded with seeds using an automated process. Alternatively, loaders may be configured to be automated in part and require manual operation in part.

### Example Carriers

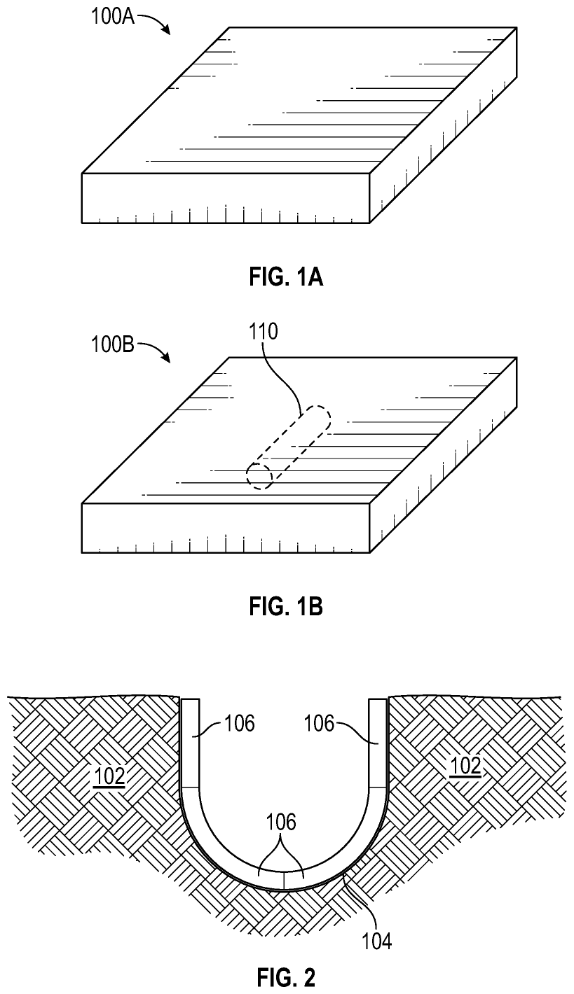

FIGS. 1A and 1B are perspective views of an example carrier 100 in a “cold” carrier 100A state and a “hot” carrier 100B state. As noted above, carriers may be available in various shapes, sizes, and configurations. The example carrier 100 is a “tile” carrier that is generally square in shape (e.g., substantially the same width and length), with a much smaller height. For example, the width and length of the carrier 100 may be orders of magnitude larger than the height of the carrier, such as a factor of 200 to 1,000. In one example, the width and length of the carrier 100 are about 1 cm, and the height of the carrier 100 is about 1 mm-8 mm. In other embodiments, carriers may have other sizes, configurations, materials, etc.

Carrier 100A is considered a “cold” carrier because it has not yet been loaded with a radioactive seed. Carrier 100B is considered a “hot” carrier because it has a radioactive seed 110 embedded within it. As discussed in the related patents and patent applications noted above, the radioactive seeds can have various shapes, sizes, and characteristics. In the examples discussed herein, radioactive seeds are illustrated as generally cylindrical, such as the cylindrical seed 110. However, other shapes and sizes of seeds may be used in other implementations. In the examples discussed herein, radioactive seeds are substantially rigid, such that they maintain their shape within their respective carriers. In other embodiments, seeds may be more pliable, such that they are somewhat malleable in taking on a shape of a specific treatment surface.

Carriers may comprise various materials, such as one or more biocompatible materials including collagen. Carriers may be configured for permanent implantation into a tumor bed, such as to provide radioactive energy to a treatment surface surrounding an area where a tumor has been removed in order to treat any remaining malignant tissue. Carriers can be composed of various materials and take on various shapes and sizes.

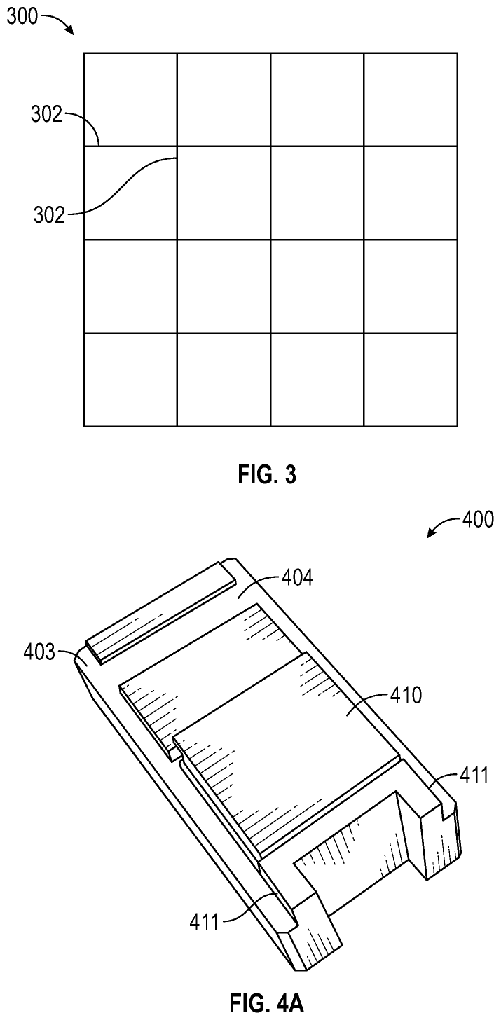

FIG. 2 is a cross-sectional drawing illustrating a portion of patient tissue 102 having a tumor bed 104 therein. The tumor bed 104 may have been created by a surgical process, such as a tumor debulking process that removed tumor cells. For example, the tissue 102 may represent cranial tissue of a human (or other mammal) wherein the tumor bed 104 is surgically created in order to remove one or more tumors from the brain (and/or surrounding areas) of the patient. Thus, the tissue 102 may represent different types of material, such as bone, fatty tissue, brain tissue, etc. Any reference herein to “tissue” may reference to any type of mammalian material, including bone, fatty tissue, brain tissue, etc.

In the example of FIG. 2, four tile carriers 106 (e.g., such as the hot carrier 100B) are illustrated as already placed within the tumor bed 104, and radiation is delivered to the treatment area by the radioactive seeds within the carriers 106. Placement of the tiles 106 in the tumor bed 104 may occur in the surgical room, such as immediately after a surgical procedure, or elsewhere at some time after the surgical procedure, such as in a separate procedure performed hours, days, or weeks later. In the embodiment of FIG. 2, the tiles 106 are pliable such that they conform to the treatment surface, which is the non-planar outer surface of the tumor bed 104 in this example. In one embodiment, the tiles 106 may comprise collagen that provides such flexibility in conforming the tiles 106 to a nonplanar surface.

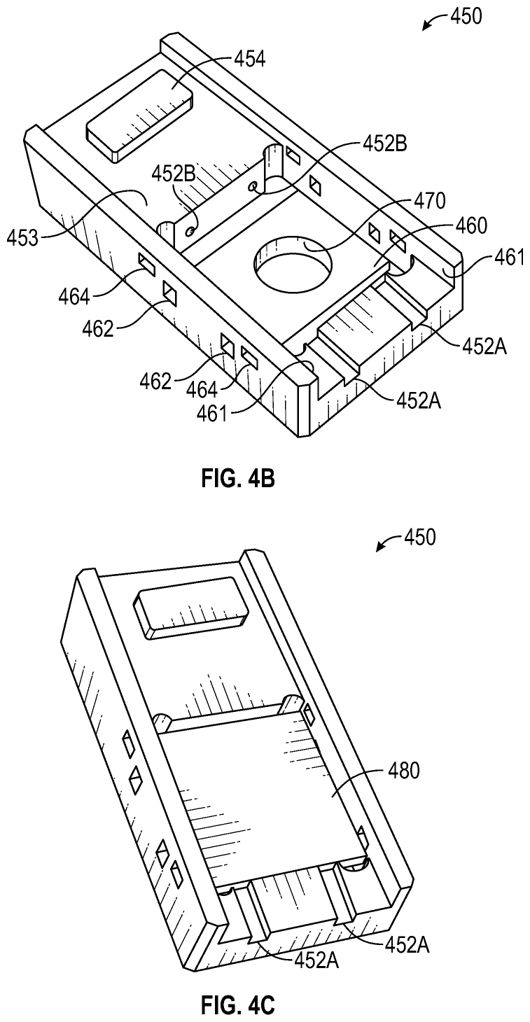

FIG. 3 is a top view of a carrier sheet 300 configured for implantation of multiple radioactive seeds. The example carrier sheet of 300 may be, for example, a 4 cm×4 cm carrier sheet configured for trimming at each centimeter to create sixteen 1 cm×1 cm carriers. In some embodiments, the carrier sheet 300 may include trim lines at the locations where trimming, or cutting, of the carrier sheet should be performed, such as the trim lines 302 positioned at one centimeter intervals. A carrier sheet may be trimmed using scissors, razor blade, knife, die cutter, or similar cutting implement, to create custom sized and/or shaped carriers.

Using the trim lines in the example carrier sheet 300, a user can accurately create multiple carriers having dimensions anywhere between 1 cm-4 cm on either side. For example, a user could create four 1 cm×4 cm carriers, two 2 cm×4 cm carriers, or sixteen 1 cm×1 cm carriers using the trim lines on carrier sheet 300. In other embodiments, trim lines may be placed at different intervals. In one embodiment, trim lines may be generated by a dosimetric planning software, such as the software discussed in U.S. patent application Ser. No. 15/017461, entitled “Radioactive Implant Planning System and Placement Guide System,” filed on Feb. 5, 2016, which is hereby incorporated by reference in its entirety. For example, in order to achieve a maximum therapeutic index, a custom shaped carrier sheet or multiple carrier sheets may be determined by software and implemented by the user. In one embodiment, the software generates a printout of the trimming pattern that can be overlaid on a carrier sheet so that the printed trimming pattern as well as the carrier sheet thereunder can be simultaneously trimmed in order to achieve the calculated carrier size, shape, pattern, etc.

### Example Loading Device and Method

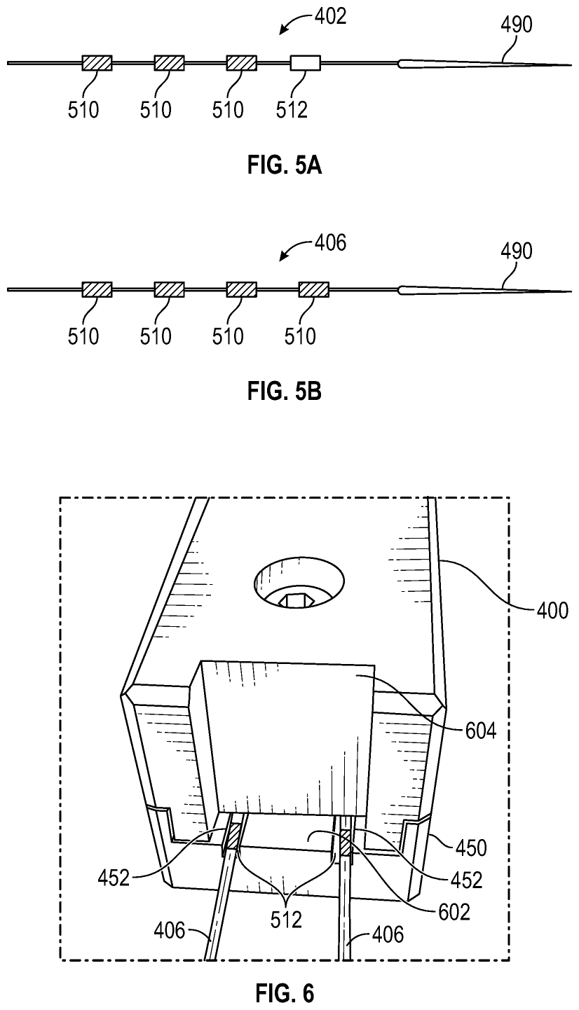

FIGS. 4A-4C are perspective views of a loading apparatus that is usable to embed radioactive seeds into carriers, while limiting exposure of the user to radioactive energy from the radioactive seeds. Advantageously, the loading apparatus facilitates accurate positioning of radioactive seeds within a carrier, as discussed in further detail below. The illustrated loaders comprise two components, a base and a lid, although in other embodiments the loaders may be separated into additional components. While specific examples of a loader are illustrated, the illustrated loaders may be modified to accommodate a wide variety of carrier dimensions and styles while still providing certain of the advantages discussed herein.

The example loaders illustrated herein are configured for facilitating embedding of radioactive seeds in conjunction with a strand of suture having one or more seeds attached thereto (e.g., seed-in-suture), such as may be inserted through the carrier using a needle, or similar insertion device. However, seeds may be inserted using the loaders discussed herein in other manners and with other insertion devices, such as to facilitate embedding of loose seeds.

In the example of FIG. 4 (including FIGS. 4A, 4B, and 4C), the loader includes a cover 400 and a base 450 configured for engagement with one another to secure a carrier sheet (or single carrier) for insertion of one or more radioactive seeds. In this example, the cover push block 410 is spring-loaded to engage with a carrier sheet positioned on a loading bed 460 of the base 450. An engagement surface 403 of the cover 400 is configured to securely engage with an engagement surface 453 of the base 450. Each of the base 450 and cover 400 have certain alignment features that allow for secure engagement of the surface of the base 450 and cover 400, which may vary from one embodiment to another embodiment of loader. In the example of FIG. 4, a protruding engagement surface 454 of the base 450 is configured to engage with a recessed engagement surface 404 of the cover 400. In other embodiments, other sizes, shapes, configurations, etc. of alignment features may be used to uniformly and securely engage the service and base of the loader.

As shown in FIG. 4C, a carrier sheet 480 is placed on the loading bed 460. With the carrier sheet 480 placed on the loading bed 460, the cover 400 may be engaged with the base 450 such that the push block 410 secures the carrier sheet 480 at a fixed position within the loader. The loader further comprises guide channels 452 configured to receive an insertion device, such as a needle threaded with a suture having multiple seeds attached thereto, and guide the insertion device through the secured carrier sheet 480 so that the seeds are properly placed within the carrier sheet 480. In the embodiment of FIG. 4, the guide channels include insertion channels 452B and exit channels 452A. Advantageously, the channels are aligned so that seeds are consistently uniformly placed within a thickness of the secured carrier sheet. While FIG. 4C illustrates a loader with two guide channels 452, in other implementations any number of guide channels may be used. For example, a loader may include from 1 to 10 (or more) guide channels.

The loader of FIG. 4 includes additional features that will be discussed further with reference to other figures. For example, the base 450 includes seed alignment windows 462 positioned so that, particularly when a user reinserts a carrier with a strand of seeds already embedded at 90° from the original loading direction (used for insertion of the strand of seeds), the user can view position of the strand of seeds within the carrier positioned within the loader. For example, in some embodiments, when a strand is positioned properly (e.g., seeds are positioned in a central portion of individual carriers that will be separated after seed implantation is completed), seeds should be viewable in one or both of the alignment windows 462 (e.g., through a portion of a collagen carrier). In other embodiments, other alignment window configurations are possible, such as use of a single alignment window or additional alignment windows. Example base 450 also includes carrier alignment windows 464, which are usable to ensure that the carrier sheet is properly seated on the loading bed 460, such as by confirming that each of the four corners of the carrier are visible in the four alignment windows 464 of base 450.

In some embodiments, the loading bed 460 may be adjustable to alter position of a carrier sheet with reference to the guide channels 452. For example, the loading bed 460 may be lowered towards a bottom of the loading device to align the guide channels 452 with an upper portion of the carrier sheet (because the carrier sheet is lower with reference to the guide channels). The loading bed 460 may be mechanically adjustable or may be adjusted using inserts that are placed on the loading bed. For example, a lowest loading bed position may align an upper portion of carriers sheets with the guide channels. An insert may be placed on the loading bed, followed by placement of the carrier sheet on the insert, to align a middle portion of carrier sheets with the guide channels. Another insert may be added on top of the first insert, followed by placement of the carrier sheet on the stacked inserts, to align a lower portion of carrier sheets with the guide channels. In this manner, the loading device is usable for insertion of seeds at multiple levels within a carrier sheet.

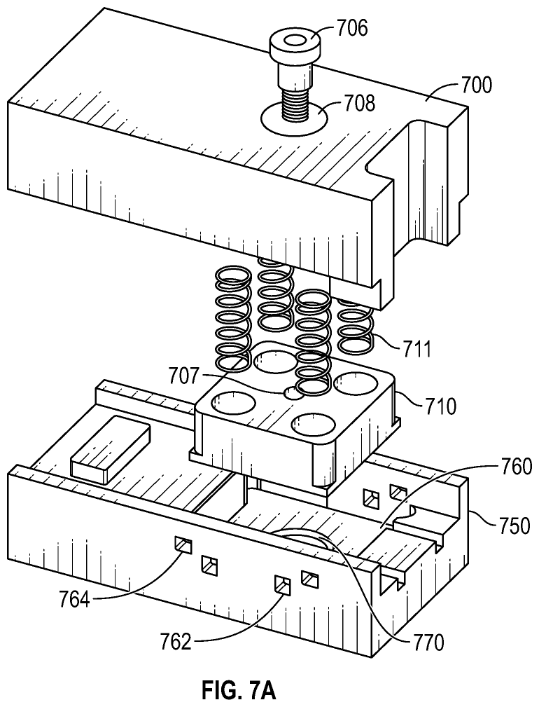

FIGS. 5A and 5B illustrate example strands 402 and 406 that may be used, in conjunction with an insertion device 490, with the loaders discussed herein. Each of the strands 402 and 406 include multiple radioactive seeds 510 attached to the strand (e.g. suture or other durable material). The strand 402 includes a dummy seed 512 that is sized and shaped similar to a radioactive seed 510, but is not radioactive itself. In other embodiments, a dummy seed may be simply a colored marker on the strand, rather than a structure size and shaped similar to a radioactive seed. In some embodiments, a dummy seed 512 may be used to determine when each of the radioactive seeds 510 are properly placed within a carrier sheet. In the embodiment of FIG. 5, the insertion device 490 is a needle, but other insertion devices may also be used.

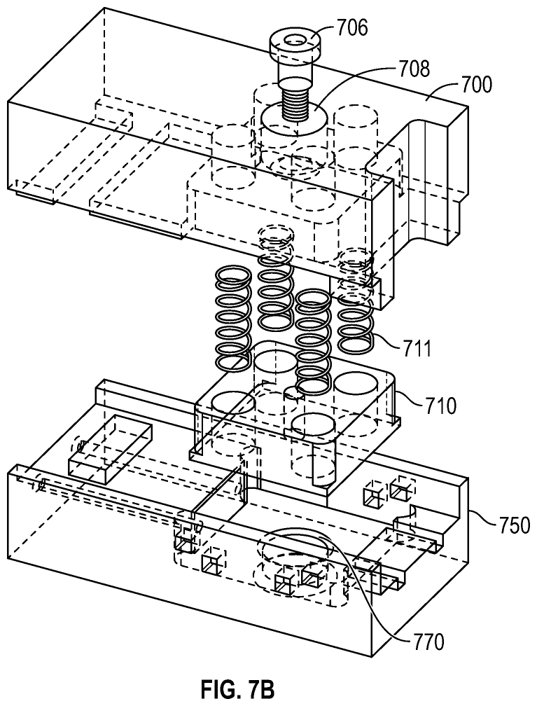

Moving to FIG. 6, a perspective view of an insertion side of a loader, comprising base 450 engaged with cover 400, is shown with strands 406 inserted through each of the guide channels 452. In this example, dummy seeds 512 are shown positioned with reference to the cover 400 to indicate that the strand, and the corresponding radioactive seeds on the strand, are properly positioned within the carrier sheet. In particular, the strand in this example is configured so that when the dummy seeds 512 are aligned with an alignment base 602, the user may determine that the radioactive seeds are properly positioned within the one or more individual carriers of a carrier sheet within the loader. In this example, the cover 400 is manufactured to include a recess 604 that defines the alignment base 602 allows when the cover 400 and base 450 are engaged, and that allows visibility of strands entering the guide channels 452 across the alignment base 602.

In some implementations, a needle longer than the loader may be used and pulled through the loading channels. Once the needle protrudes from the loader, it may be pulled the rest of the way with clamps, needle-nose plier, or another tool (such as an automated robotic tool). For example, if a loader length is 10 cm, an 11 cm (or longer) needle may be used to feed the strand through the loading channels and embed the seeds within the carrier sheet.

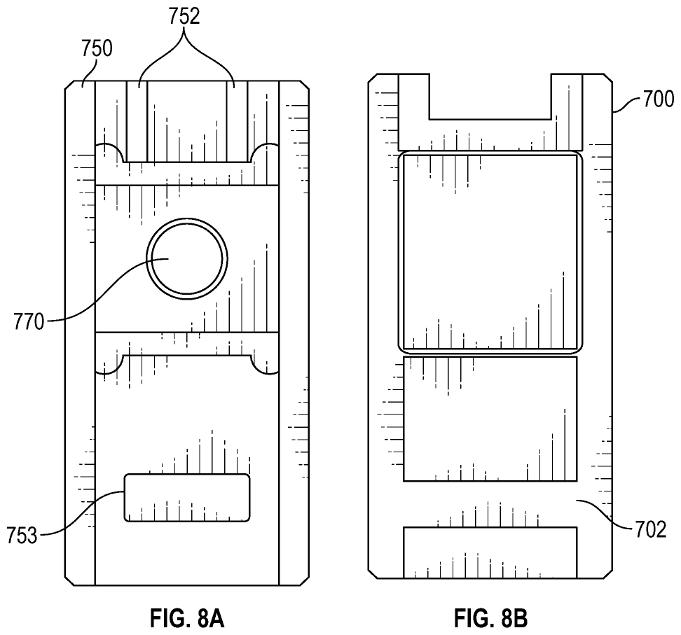

FIGS. 7A and 7B are exploded views of a loader, each including a cover 700, a base 750, and push block 710. FIG. 7B includes broken lines illustrating portions of the loader not viewable from the perspective view shown in FIG. 7A. In this embodiment, a shoulder screw 706 threadedly engages with threaded recess 707. The shoulders screw 706 maintains attachment of the push block 710 with the cover 700, so that the springs 711 are substantially retained within the push block 710. The push block 710 maintains mobility by virtue of a movement channel 708 that allows the shoulder screw 706 to move up and down within the channel 708 as the push block 710 is moved up and down.

Also visible in FIG. 7 is an access port 770 through a bottom surface of the base 750. In some embodiments, the access port 770 is usable for removal of a carrier sheet from the loader. For example, a push rod, screwdriver, tweezers, etc. may be inserted into the aperture 770 to push the carrier sheet off the loading bed surface, allowing easy removal of the carrier sheet from the loader. In some embodiments, a bidirectional plunger, or other mechanical structure may be attached to the base 750 around the aperture 770 for the same purpose, while retaining attachment to the base 750.

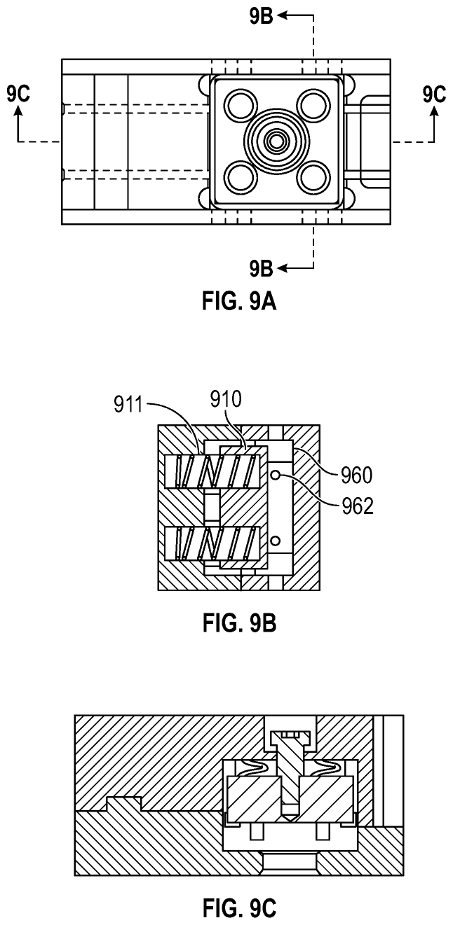

FIG. 8A illustrates engagement surfaces of the base 750 and cover 700. As shown in FIG. 8B, which illustrates the cover 700, the engagement surface 702 of the cover 700 is configured to securely engage with an engagement surface 753 of the base 750 shown in FIG. 8B. Also viewable in FIG. 8A is the access port 770 through which a tool may be inserted to remove a carrier sheet. In this embodiment, loading channels 752 are part of the base 750, but in other embodiments the loading channels may be entirely or partially part of the cover 700.

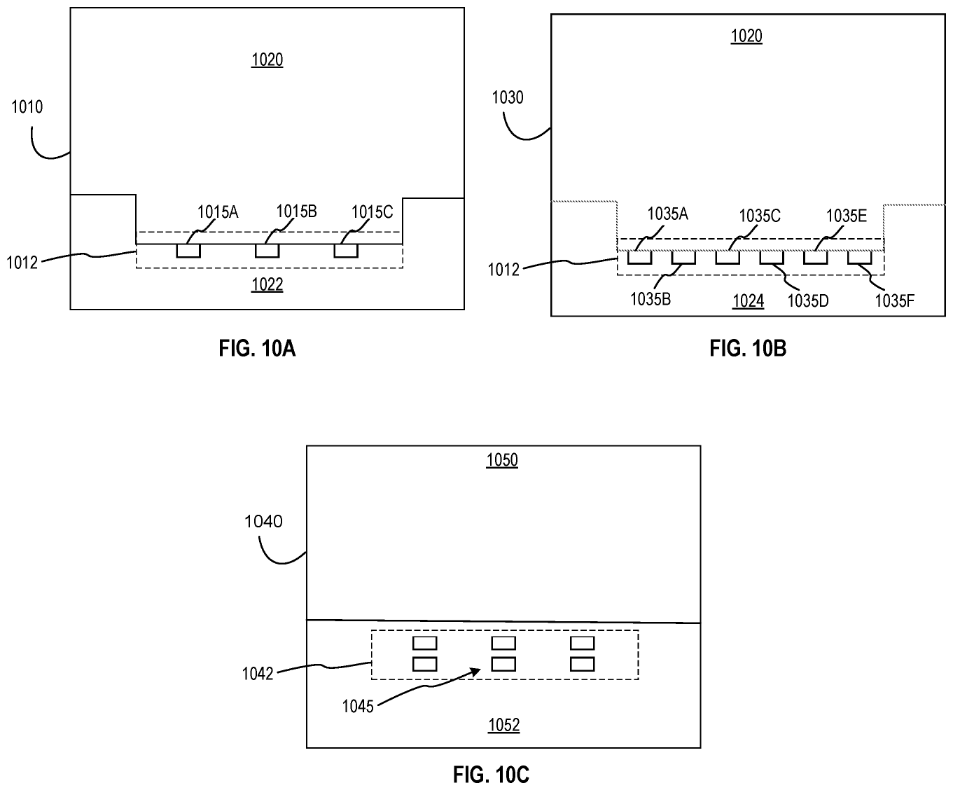

FIG. 9A is a top view of a loader, with FIG. 9B illustrating a cross-section 9B of FIG. 9A and FIG. 9C illustrating cross section 9C of FIG. 9A. FIG. 9B illustrates the springs 911 in a compressed position and push block 910 against the loading bed 960. Loading channels 962 are shown in FIG. 9B, which are configured to receive a needle pulling a strand of seeds through the loader and into a carrier positioned on the loading bed 960.

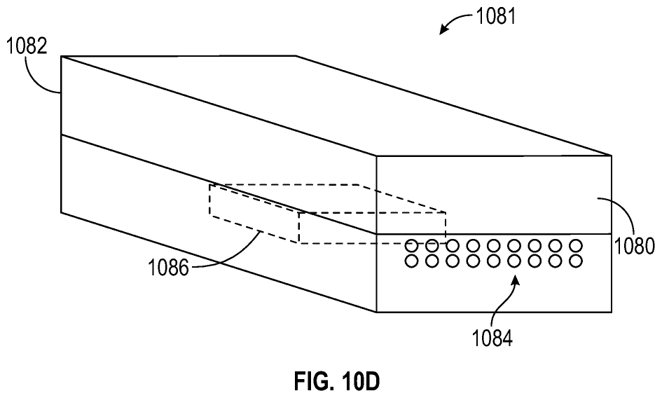

FIGS. 10A-10B are end views of example loading devices, with varying quantities and positions of guide channels. The end views in FIGS. 10A and 10B are a distal end of the loading devices, that is, the end where the needle (or other seed introduction device) exits the loading device. In some embodiments, the quantity of guide channels may vary, such as to provide the operator more flexibility in where radioactive seeds are positioned within a carrier sheet placed within the loading device. In addition to allowing flexibility in positioning and quantity of seeds in a single horizontal axis (e.g., aligned with a middle of the carrier sheets height), in certain embodiments loading devices include guide channels that allow loading radioactive seeds at various vertical levels within a carrier sheet, such as at a top of the carrier sheet, a middle of the carrier sheet, and/or at the bottom of the carrier sheet. The examples in FIG. 10 are illustrative of the general structure and concept for allowing additional guide channels, but many other variations of the particular sizes, positions, etc. of the loading devices and guide channels are contemplated. For example, the various components of the example loading devices and carrier sheets are not necessarily drawn to scale, but instead some components may be shown larger (or smaller) in the drawings for illustrative purposes. Additionally, multiple guide channels, such as in a single row, multiple rows, or offset in any other manner, may be used in conjunction with any other loading device discussed herein, such as in place of a single or double guide channel embodiment.

FIG. 10D is a perspective view of a proximal end 1080 of a loading device 1081, showing a plurality of loading channels 1084 where a seed introduction device, such as a needle or pushrod, may be inserted into the loading device, through the carrier 1086 positioned within the loading device, and out the distal end 1082 of the loading device.

Returning to FIG. 10A, a distal end of a loading device 1010 is shown, including a cover 1020 and a base 1022. A carrier sheet 1012 is shown as it would be positioned within the closed loading device. The embodiment of FIG. 10A includes three exit guide channels 1015A, 1015B, and 1015C. In one embodiment, the carrier sheet 1012 includes a grid of tile carriers that will be cut apart after loading with radioactive seeds. For example, a 3×3 grid of radioactive carriers may be included in the carrier sheet 1012. Thus, a strand with three radioactive seeds may be inserted into guide channels on a proximal end of the loading device (not shown) and pulled (or pushed) through the guide channel until the three radioactive seeds are in the center of the first three tile carriers and the seed introduction device exits through guide channels 1015A-1015C. In this manner, nine tile carriers may be loaded while the carrier sheet is within the loading device 1010.

In the example of FIG. 10B, the loading device 1030 includes the same cover 1020, but a base 1024 that includes six exit guide channels 1035A-1035F (that may correspond with six entry guide channels on a proximal end of the loading device. In this embodiment, the six guide channels 1035 allow the user more flexibility in where radioactive seeds are positioned within the carrier sheet 1012. As noted above, in some embodiments the loading bed of the base 1022 or 1024 may be adjustable, such as to raise or lower the carrier sheet 1012 to a position where the guide channels 1015 or 1035 are aligned with a higher or lower portion of the carrier sheet 1012, for example. Additionally, in some embodiments the loading bed may be adjustable while the carrier sheet is inside the loading device, such that the loading bed may position the carrier sheet for insertion of one or more radioactive seeds on an upper portion of the carrier sheet, and then raise the loading bed for placement of one or more radioactive seeds on a lower portion of the carrier sheet.

FIG. 10C illustrates a proximal end of a loading device, that is, the end where the needle (or other seed introduction device) enters the loading device. In this embodiment, the entry guide channels are rectangular, similar to the shape of the exit guide channels discussed with reference to FIGS. 10A and 10B. In other embodiments, the entry (and/or exit) guide channels may be any other shape. For example, the entry guide channels may be circular, such as discussed below, sized to provide support and guidance for a needle (or other seed introduction device). In other embodiments, the guide channels may be shaped as triangles, hexagon, or any other shape. In some embodiments, the shape is non-uniform, e.g., one or more sides of the channels are keyed, such as a flat edge of an otherwise round circle or a protrusion from one side of the channel, to match a shape of a specially designed seed introduction tool. In such an embodiment, rotation of the seed insertion device is limited.

FIGS. 10E-10G illustrate proximal ends of example loading devices 1040, 1060, and 1070, respectively, that each include multiple rows of guide channels. In these embodiments, the multiple rows of guide channels allow the user flexibility in positioning of radioactive seeds at different depths of the carrier, such as at the top portion, a central portion, and/or a bottom portion of a carrier sheet. In the example of FIG. 10E, six entry guide channels 1045 are illustrated in two horizontal rows and three vertical columns. In the example of FIG. 10F, eighteen entry guide channels 1065 are shown, in two horizontal rows and 9 vertical columns. Finally, in FIG. 10G, a four by fourteen grid of guide channels 1065 is shown in the base 1056. Thus, the carrier sheet 1072, which may include multiple tile carriers or other shapes or sizes of carriers, may receive a seed insertion device at up to 56 different positions on the end of the carrier sheet 1072, or even more positions in an embodiment where the loading bed is adjustable up and down.

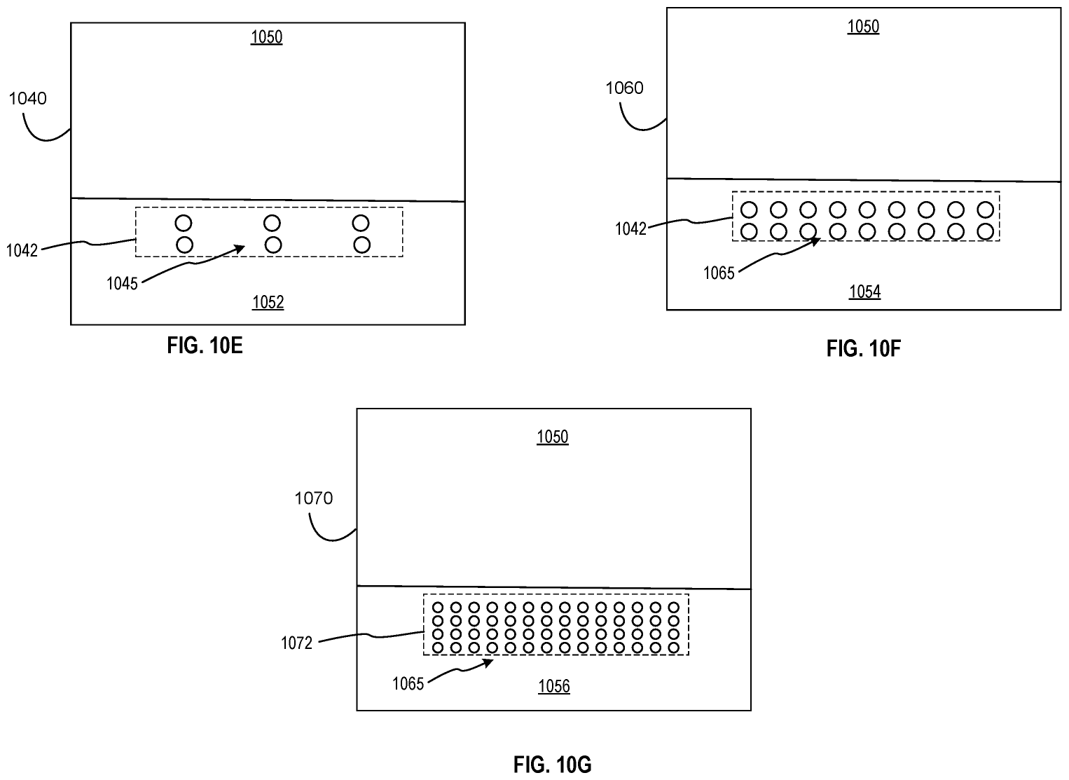

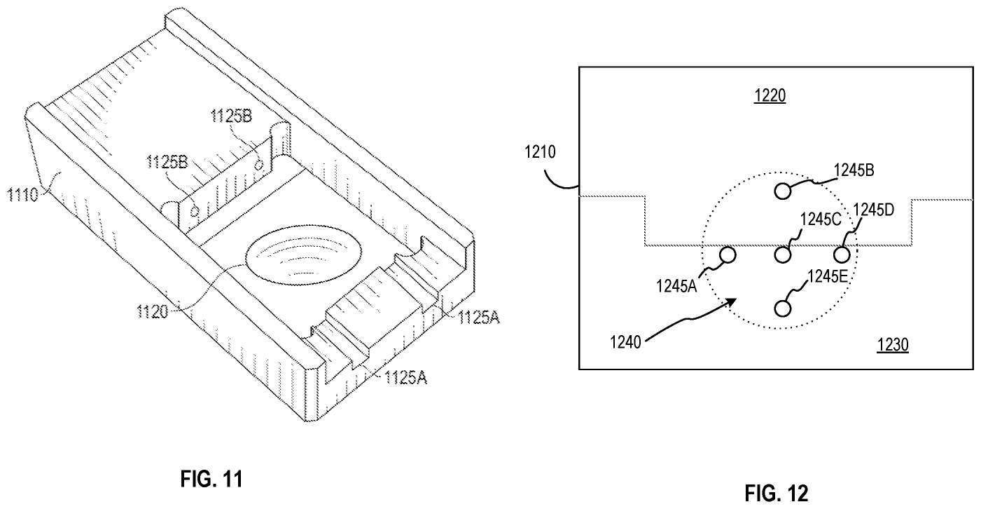

FIG. 11 is a perspective view of a loader base 1110 that may be used to retain spherical, or semi-spherical, carriers for insertion of radioactive seeds. As shown, the loading bed of the base 1110 includes a hemispherical cut out 1120 sized to receive a spherical radioactive seed carrier. Although not shown, a cover with a similar hemispherical cut out may be positioned atop the base 1110 (with the cut out 1120 and the cut out of the cover aligned with the carrier) to more securely maintained in position the spherical carrier as one or more radioactive seeds are inserted into the carrier via an insertion device (e.g., a needle) extending through a guide channel 1125B, through a portion of the carrier sitting within the cutout 1120, and into an exit guide channel 1125A. In other embodiments, other quantities and positions of guide channels are possible. For example, FIG. 12 is a proximal end view of a loading device 1210 configured for loading of radioactive seeds within a spherical, or hemispherical, carrier. In this example, a base 1230 includes a hemispherical cut out, such as cutout 1120 in FIG. 11, sized to receive a spherical carrier 1240, shown here as positioned within the loading device 1210. In this example, the base 1230 includes guide channels 1245A, 1245D, and 1245E around a periphery of the spherical carrier, with guide channel 1245C at a center of the spherical carrier. In this example, the lid 1220 includes a hemispherical cut out sized to engage with an upper portion of the spherical carrier 1240. The lid 1220 also includes one guide channel 1245B positioned to insert a radioactive seed within an upper portion of the spherical carrier 1240. In other embodiments, other quantities and positions of a channels may be used.

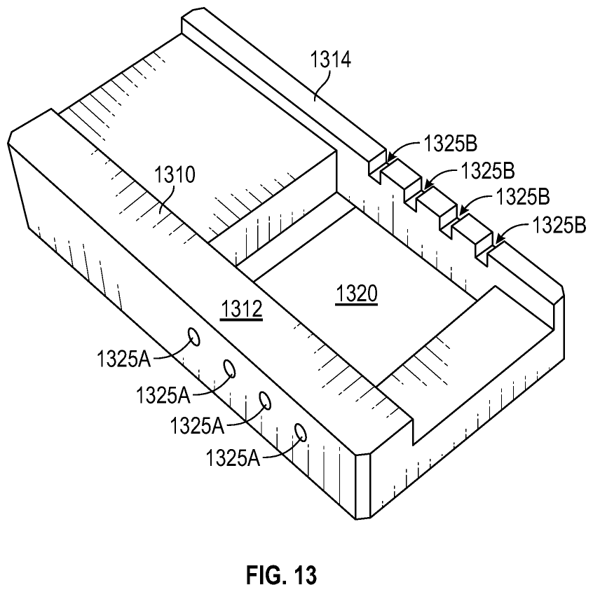

FIG. 13 is another example base 1310, which may be used in conjunction with a corresponding lid (not shown) to retain a carrier sheet (or other shape carrier) for insertion of one or more radioactive seeds. In the example of FIG. 13, entry guide channels 1325A and exit guide channels 1325B are on opposite sides of the base 1310. Thus, a radioactive seed introduction device (e.g., a needle or push rod) may be inserted through any of the entry guide channels 1325A, through a carrier sheet positioned on the loading bed 1320, and out the corresponding exit guide channels 1325B. In this embodiment, the exit guide channels 1325B are open so that a thread or suture that is used for loading a string of radioactive seeds into a carrier sheet is able to be lifted out of the loading device along with the loaded carriers. In some embodiments, guide channels may be included on the ends of the loading device, as in FIG. 10, and/or on the sides of the loading device, as in FIG. 13.

In other embodiments, different quantities and/or positions of guide channels may be used in a loading base and/or loading cover. In the example of FIG. 13, the entry side 1312 of the base 1310 is widened to provide additional directional support of a seed introduction device as it passes into the loading chamber and the carrier sheet, and decreases the complexity of aligning the seed introduction tool with the corresponding exit guide channel 1325B on exit side 1314. In other embodiments, the sides may be equally size.

### Example Loader Variations and Options

Depending on the embodiment, a loader may be a sterilizable single or multi-use device for manual or automated loading (in real time or for pre-loading) of carriers such as but not limited to tiles, gores, or stars with radioactive seeds such as I¹²⁵, Cs¹³¹, Pd¹¹¹, Pd¹⁰³ and/or other materials. The loaders may be constructed of metal, plastic or composite material, and manufactured by machining, casting, molding, stamping, forming or 3D printing. Embodiments of the loaders contemplated may include shielding either by way of construction with a high Z material, or with other materials with a sufficient dimension (thickness) to provide the necessary dose attenuation for a user. Other embodiments may remain unshielded, and be made of materials suitable for the purpose including but not limited to tungsten, stainless steel, nylon or plastic.

In some embodiments, an automated system may include one or more automate movement of one or both the of the main loader components, e.g., the base and cover, to automate engagement of the base on cover surface for loading of seeds. Automation of carrier sheet placement on the loading bed as well as automation of insertion of an insertion device with the string of seeds may also be performed. Such automations may be accomplished through use of hydraulic, electronic, and/or manual energy.

### Example Transparent Loader Surfaces

In some embodiments, a loader may include one or more surfaces (e.g., one or more portions of the various bases, covers, push blocks, windows, etc.) composed of a transparent material, where transparency is defined to include embodiments with various levels of transparency, from fully transparent to partially transparent. In some embodiments, a level of transparency is determined or selected to provide suitable visibility to a radioactive seed position behind the transparent surface. Thus, depending on the embodiment, the level of transparency required to provide this advantage may vary. For example, if the seed location is detected by human vision, such as by a technician or surgeon that manually handles the loader and monitors position of the seed within the loader, the transparency level may be higher than if the seed location is monitored with a digital detector (e.g., a camera or sensor) that monitors and automatically detects light, whether in the visible or invisible spectrum (e.g., with object detection software executing on a computing device).

In some embodiments, the level of transparency may also be determined or selected to provide suitable shielding of radioactive energy from radioactive seeds or other sources that are placed behind the transparent surface. For example, in some embodiments the transparent material may be impregnated with lead or another material that absorbs or reflects radioactive energy. In embodiments where a human user is handling the loader, the level of radioactive shielding may be increased to reduce radiation absorbed by the user's tissue. Conversely, in a loader system where seeds are inserted into carriers using robotic components, for example, radioactive shielding requirements may be reduced.

Depending on the embodiment, the transparent loader surfaces may be formed of various materials, such as glass, plastic, etc. Such materials may allow shielding objects to be implanted within the material, such as via an injection molding process. Depending on the embodiment, shielding material may be formed above, below, or within a transparent surface of a loader in various manners. For example, in some embodiments a thin layer of high Z foil may be placed above and/or below a transparent surface, such as across an entire area of the transparent surface or a portion of the transparent surface. For example, a suitable shielding level may be obtained by use of a shielding material across the portion of a transparent surface of a loader, while still providing a suitable level of visibility beyond the transparent surface.

### Overview of Certain Technical Improvements

Below are several improvements in the various loaders discussed herein. Depending on the particular loader implementation, some or all of these advantages may be present in a particular loader.

Spring loaded cover pushes and keeps the carrier sheet in place on base. For example, with reference to FIG. 7, when the cover 700 is engaged with the base 750, the push block 410 will be pushed against the loading bed 760 by the springs 711. When the carrier sheet is placed on the loading bed 760, the push block 410 exerts force on the carrier sheet so that it maintains a stable position between the push block 710 and the loading bed 760, allowing an insertion device to pass through a thickness of the carrier sheet without causing the carrier sheet to fold, buckle, or bend. Advantageously, this spring loaded cover allows carrier sheets of various thicknesses to be used in the same loader, since the force of push block 410 is not locked into a particular position.

Carrier windows allow manual or automatic (e.g., machine vision) confirmation of proper placement of a carrier sheet in the loader. For example, with reference to FIG. 4, the carrier alignment windows 464 allow a user to see within the loading bed at a height just above where the carrier sheet should be positioned to ensure that the carrier sheet is properly positioned and of the correct thickness prior to inserting the radioactive seeds. As noted above, the windows may be apertures and/or may include material that allows at least partial transparency, while possibly also provided some shielding. In some embodiments, an optical sensor may be positioned to view the carrier alignment windows and detect whether the carrier sheet is properly positioned.

Seed alignment windows allow manual or automatic (e.g., machine vision) confirmation of proper placement of seeds within the carrier sheet prior to removing the carrier sheet from the loader. For example, with reference to FIG. 4, the seed alignment windows 462 allow a user to see within the loader at the position of the carrier sheet, such as when the carrier sheet is rotated 90° and reinserted into the loader, to determine whether the radioactive seeds are properly positioned within the carrier sheet. For example, the seed alignment windows 464 may be positioned to align with a first and/or last seed when the strand is properly positioned within the carrier. As noted above, the windows may be apertures and/or may include material that allows at least partial transparency, while possibly also provided some shielding. In some embodiments, an optical sensor may be positioned to view the carrier windows and detect whether the carrier sheet is properly positioned.

Cutout of lid (or base) to view marker on strand indicating proper position of seeds in carrier. For example, as illustrated in FIG. 6, dummy seeds 512, or simply a marking 512 on the strand, may be aligned with the alignment based 602 when the strand is properly positioned. In some embodiments, a dummy seed 512 may be properly positioned when it starts to exit the loader, has just entirely exited the loader, or other known position that indicates proper placement of the seeds within the carrier sheet.

Guide channel(s) through length of base (and/or cover) for guiding needle and strand of seeds. As discussed above, the guide channels may be positioned in one or both of the base and cover depending on the embodiment. Advantageously, the guide (or “loading”) channels are configured to maintain the insertion device and strand of seeds in a uniform path as it passes through the carrier sheet, such as so that each of the seeds our positioned at a central, or predetermined offset, depth of the carrier sheet. For example, in some implementations, such as according to a particular dosimetric plan, seeds are positioned closer to one surface of the carrier sheet than the other, such as 1 mm from a treatment surface of a 4 mm thick carrier sheet. Thus, the guide channels are carefully calibrated to ensure that each of the seeds is positioned at the precise offset within the carrier sheet.

Access port on base below loading bed allows carrier to be pushed out of loader. For example, FIG. 4B illustrates an access port 470 through which a tool may be inserted to push a carrier service from the loading bed, such as after seeds have been loaded and the cover 400 has been removed from the base 450.

Alignment features provide more secure interface between cover and base. For example, FIGS. 4A and 4B illustrate alignment features including engagement surface 454 that engages with surface 404, and outer surfaces 411 of cover 400 that engage with inner surfaces 461 of the base 450.

Although certain embodiments have been described with reference to the above examples, it will be understood that modifications and variations are encompassed within the spirit and scope of the invention.

### Other Embodiments

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments of the invention. It will be appreciated, however, that no matter how detailed the foregoing appears in text, the invention can be practiced in many ways. As is also stated above, the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A loading apparatus comprising:
a loading bed configured to support a radioactive seed carrier configured to receive one or more radioactive seed; and
at least one guide channel configured to receive an injection device, the injection device configured to inject the radioactive seed into the radioactive seed carrier, wherein the loading bed is movable with reference to the at least one guide channel.

2. The loading apparatus of claim 1, wherein the loading bed includes a cavity shaped to secure at least a bottom portion of the radioactive seed carrier.

3. The loading apparatus of claim 2, wherein the cavity is semi hemispherical and the radioactive seed carrier is spherical.

4. The loading apparatus of claim 1, wherein the loading apparatus further comprises a lid configured to exert force on the radioactive seed carrier towards the loading bed.

5. The loading apparatus of claim 4, wherein the lid comprises a semi hemisherical cavity configured to engage with an upper portion of the spherical radioactive seed carrier.

6. The loading apparatus of claim 1, wherein the loading bed is movable after the radioactive seed is injected into the radioactive seed carrier, wherein at least one guide channel is positioned to support insertion of the injection device into the radioactive seed carrier at a different vertical position.

7. The loading apparatus of claim 1, wherein the at least one guide channel is circular.

8. The loading apparatus of claim 8, wherein the at circular guide channel has a diameter that is substantially the same as a diameter of the injection device.

9. The loading apparatus of claim 1, wherein the at least one guide channel includes at least one entry guide channel on a proximal end of the loading apparatus and at least one exit guide channel on the distal end of the loading apparatus.

10. The loading apparatus of claim 9, wherein the at least one guide channel further includes at least one entry guide channel on a first side of the loading apparatus and at least one exit guide channel on an opposite second side of the loading apparatus.

11. The loading apparatus of claim 9, wherein the at least one entry guide channel is circular and the at least one exit guide channel is open-ended upwardly.

12. The loading apparatus of claim 1, wherein the at least one guide channels include at least two rows of guide channels.

13. The loading apparatus of claim 1, wherein a height of the loading bed is adjustable by inserting one or more spacers on the loading bed.
